(19) United States
(12) Patent Application Publication
(10) Pub. No.: US 2019/0046261 A1
(43) Pub. Date: Feb. 14, 2019

(54) ENERGY TREATMENT INSTRUMENT

(71) Applicant: OLYMPUS CORPORATION, Tokyo (JP)

(72) Inventor: Shoei TSURUTA, Tokyo (JP)

(73) Assignee: OLYMPUS CORPORATION, Tokyo (JP)

(21) Appl. No.: 16/163,723

(22) Filed: Oct. 18, 2018

Related U.S. Application Data
(63) Continuation of application No. PCT/JP2016/064172, filed on May 12, 2016.

(51) Int. Cl. A61B 18/14; A61B 18/08; A61B 18/02

(52) U.S. Cl. CPC A61B 18/1445; A61B 18/02; A61B 18/085

(57) ABSTRACT

An energy treatment instrument includes: a first jaw including a first holding surface including a first end region, a second end region and a first reference position; a second jaw including a second holding surface including a third end region, a fourth end region and a second reference position; a first electrode disposed in the first end region; and a second electrode disposed in one of the second end region and the fourth end region, electrical current being applied between the first electrode and the second electrode. In a state in which the first holding surface and the second holding surface face each other, a clearance between the first holding surface and the second holding surface is continuously changed toward the first reference position and the second reference position and a clearance between the first reference position and the second reference position is greatest.

### CROSS-REFERENCE TO RELATED APPLICATION

This application is a continuation of International Application No. PCT/JP2016/064172, filed on May 12, 2016, the entire contents of which are incorporated herein by reference.

### BACKGROUND

The present disclosure relates to an energy treatment instrument.

In the related art, there is a known energy treatment instrument that performs treatment (join (or, anastomose) and cut off, etc.) on biological tissue by holding the biological tissue by a pair of jaws and applying energy to the biological tissue (apply a high frequency current to the biological tissue) (for example, see Japanese National Publication of International Patent Application No. 2010-527704).

Japanese National Publication of International Patent Application No. 2010-527704 discloses various structures for applying a high frequency current to the width direction of the jaws.

For example, as a first structure, in one of the jaws of the paired jaws (hereinafter, referred to as a first jaw), on a first holding surface that holds biological tissue with the other jaw (hereinafter, referred to as a second jaw), a first electrode is provided on one end side of the width direction of the first holding surface. Furthermore, on a second holding surface that is provided in the second jaw and that holds the biological tissue with the first holding surface, a second electrode is provided on the other end side of the width direction of the second holding surface. Namely, the first and the second electrodes are provided at the positions that are shifted in the width direction so as not to face each other in the closed state of the first and the second jaws. Then, by supplying high frequency electrical power to the first and the second electrodes, the high frequency current is applied, in the width direction of the jaws, to the biological tissue held by the first and the second jaws.

Furthermore, for example, as a second structure, on the first holding surface, the first electrode is provided on one end side of the width direction of the first holding surface. Furthermore, on the first holding surface, the second electrode is provided on the other end side of the width direction of the first holding surface. Then, by suppling high frequency electrical power to the first and the second electrodes, the high frequency current is applied, in the width direction of the jaws, to the biological tissue held by the first and the second jaws.

When using the structure in which a high frequency current is applied to the width direction of the jaws described above, because the portion in which the high frequency current is applied between the first and the second electrodes may be used as a heat generating portion, it is possible to limit the treatment target tissue of the biological tissue to the region closer to the center of the width direction of the jaws (between the first and the second electrode). Consequently, in the biological tissue, it is possible to reduce the effect of heat exerted on the peripheral tissue that is located on the outer side of the width direction of the jaws and that is present around the treatment target tissue and, thus, it is possible to avoid a decrease in natural healing power of the peripheral tissue.

### SUMMARY

An energy treatment instrument according to one aspect of the present disclosure includes: a first jaw that includes a first holding surface, the first holding surface including a first end region, a second end region that is separated from the first end region, and a first reference position that is located between the first end region and the second end region; a second jaw that includes a second holding surface that holds biological tissue with the first holding surface, the second holding surface including a third end region formed by projecting the first end region onto the second holding surface in a state in which the first holding surface and the second holding surface face each other, a fourth end region formed by projecting the second end region onto the second holding surface in the state in which the first holding surface and the second holding surface face each other, and a second reference position formed by projecting the first reference position onto the second holding surface in the state in which the first holding surface and the second holding surface face each other; a first electrode that is disposed in the first end region of the first holding surface; and a second electrode that is disposed on one of the second end region of the first holding surface and the fourth end region of the second holding surface, electrical current being applied between the first electrode and the second electrode, wherein on the first holding surface and the second holding surface, in the state in which the first holding surface and the second holding surface face each other, a clearance between the first holding surface and the second holding surface is continuously changed toward the first reference position and the second reference position and a clearance between the first reference position and the second reference position is greatest.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an energy treatment system according to a first embodiment;

FIG. 2 is a diagram illustrating a holding portion illustrated in FIG. 1;

FIG. 3 is a diagram illustrating the holding portion illustrated in FIG. 1;

FIG. 4 is a diagram illustrating an operation of the energy treatment system illustrated in FIG. 1;

FIG. 5 is a diagram illustrating a holding portion of an energy treatment instrument according to a second embodiment;

FIG. 6 is a diagram illustrating a holding portion of an energy treatment instrument according to a third embodiment;

FIG. 7 is a diagram illustrating a holding portion of an energy treatment instrument according to a fourth embodiment;

FIG. 8 is a diagram illustrating a holding portion of an energy treatment instrument according to a fifth embodiment;

FIG. 9 is a diagram illustrating a holding portion of an energy treatment instrument according to a sixth embodiment; and

FIG. 10 is a diagram illustrating a holding portion of an energy treatment instrument according to a seventh embodiment.

### DETAILED DESCRIPTION

In the following, embodiments will be described with reference to drawings. The present disclosure is not limited to the embodiments described below. Furthermore, components that are identical to those in the drawings are assigned the same reference numerals.

### First Embodiment

Schematic Configuration of an Energy Treatment System

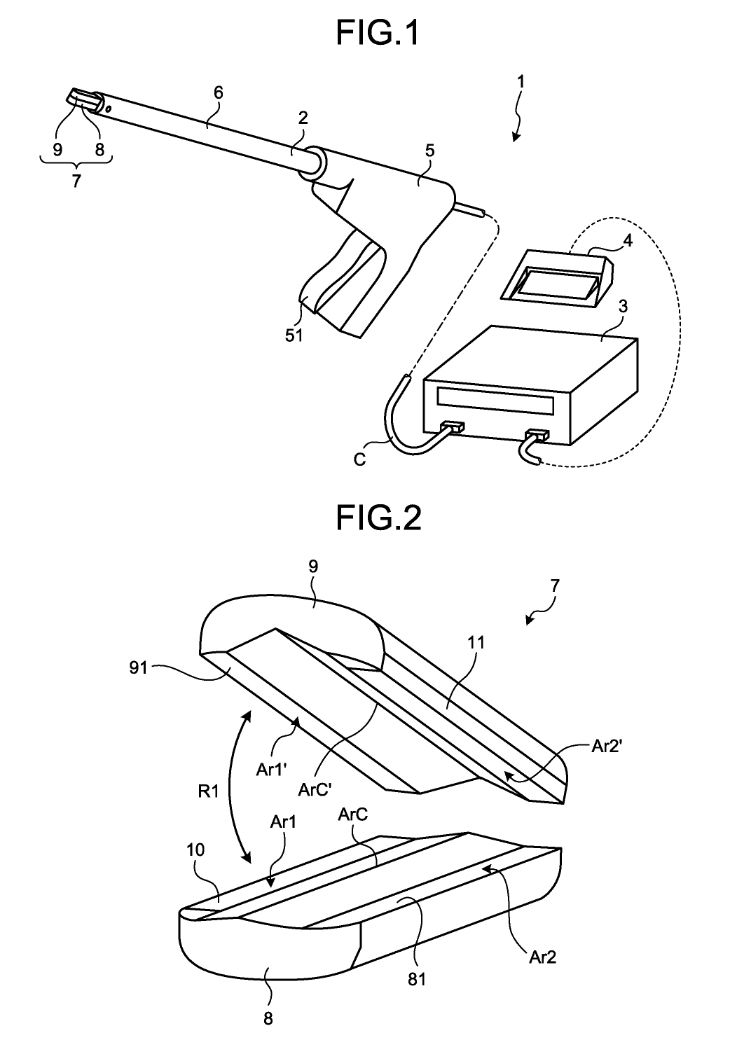

FIG. 1 is a diagram illustrating an energy treatment system 1 according to a first embodiment.

The energy treatment system 1 performs treatment (join (or, anastomose) and cut off, etc.) on biological tissue by applying energy (in the first embodiment, electrical energy (high frequency energy)) to the biological tissue. The energy treatment system 1 includes, as illustrated in FIG. 1, an energy treatment instrument 2, a control device 3, and a foot switch 4.

Configuration of the Energy Treatment Instrument

The energy treatment instrument 2 is, for example, a linear-type surgical medical treatment instrument used to perform treatment on biological tissue through an abdominal wall. The energy treatment instrument 2 includes, as illustrated in FIG. 1, a handle 5, a shaft 6, and a holding portion 7 (holder).

The handle 5 is a portion in which an operator holds the energy treatment instrument 2. Furthermore, as illustrated in FIG. 1, an operation knob 51 is provided on the handle 5.

As illustrated in FIG. 1, the shaft 6 has a substantially cylindrical shape and one end thereof (the right end portion in FIG. 1) is connected to the handle 5. Furthermore, the holding portion 7 (the left end portion in FIG. 1) is attached to the other end of the shaft 6. Then, an opening/closing mechanism (not illustrated) that opens and closes, in accordance with the operation of the operation knob 51 performed by an operator, a first and a second jaws 8 and 9 (FIG. 1) that constitute the holding portion 7 is provided inside the shaft 6. Furthermore, an electric cable C (FIG. 1) connected to the control device 3 is disposed inside the shaft 6 from one end side (on the right end side in FIG. 1) to the other end side (on the left end side in FIG. 1) via the handle 5.

Configuration of the Holding Portion

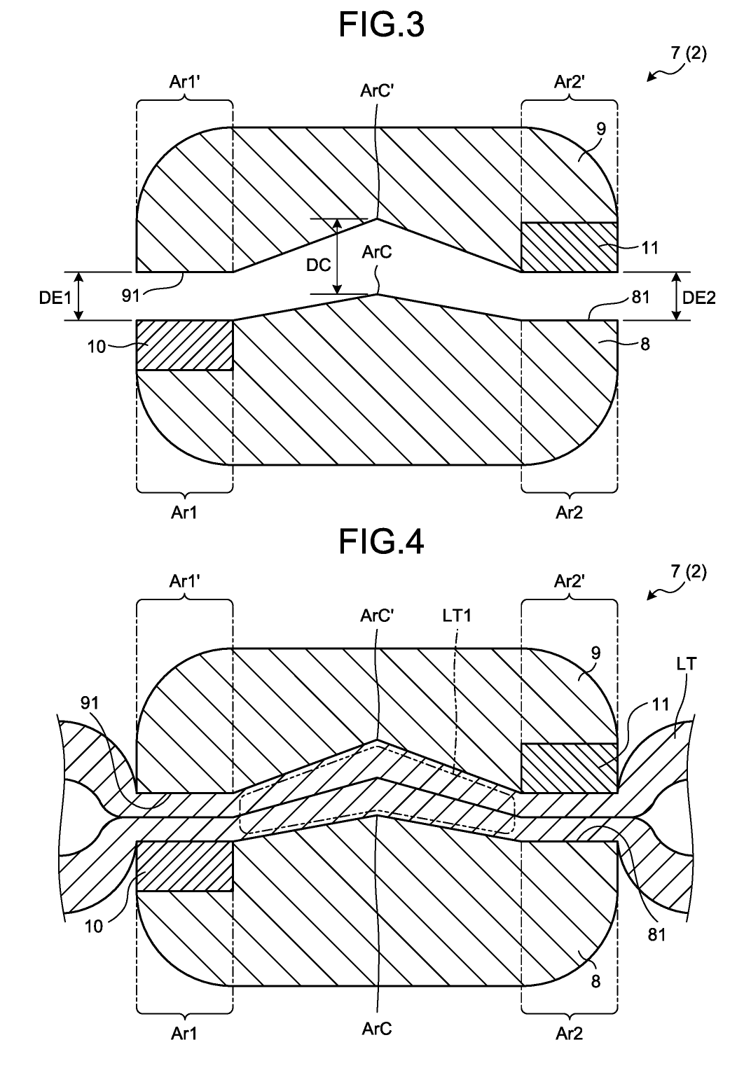

FIG. 2 and FIG. 3 are diagrams each illustrating the holding portion 7 illustrated in FIG. 1. Specifically, FIG. 2 is a perspective view illustrating the holding portion 7 that is set in the open state (in the state in which the first and the second jaws 8 and 9 are opened (separated)). FIG. 3 is a sectional view illustrating the holding portion 7 that is set in the closed state (in the state in which the first and the second jaws 8 and 9 are closed (face each other)) viewed at a sectional plane taken along the width direction of the holding portion 7 (the longitudinal direction in FIG. 3).

The holding portion 7 is a portion that holds biological tissue and performs treatment on the biological tissue. The holding portion 7 includes, as illustrated in FIG. 1 and FIG. 3, the first and the second jaws 8 and 9.

The first and the second jaws 8 and 9 are axially supported by the other end of the shaft 6 so as to be opened and closed in the direction of an arrow R1 (FIG. 2) and may hold the biological tissue in accordance with the operation of the operation knob 51 performed by the operator.

Configuration of the First Jaw

The first jaw 8 is disposed on the lower side of the second jaw 9 illustrated in FIG. 1 and FIG. 2 and has a substantially rectangular block shape extending along the central axis of the shaft 6. Examples of a material of the first jaw 8 include a material with high heat resistance and excellent electrical insulation, for example, engineering plastic, such as a polyether ether ketone (PEEK) resin. Furthermore, the material of the first jaw 8 is not limited to engineering plastic, such as a PEEK resin, but a material with non-conductive and low thermal conductivity, such as a fluorocarbon resin, and a ceramic material with low thermal conductivity, such as alumina and zirconia, may also be used. Furthermore, in addition to the above, an appropriate coating material, for example, an organic coating material, such as a fluorocarbon resin having non-adhesive property with respect to a living body, and an inorganic coating material, such as silicon, may also be added.

The surface on the upper side of the first jaw 8 illustrated in FIG. 2 and FIG. 3 functions as a first holding surface 81 that holds the biological tissue with the second jaw 9.

Here, on the first holding surface 81, the region that is located on one end side of the width direction (on the left end side in FIG. 2 and FIG. 3) and that covers the overall length of the first holding surface 81 (the overall length in the longitudinal direction, the same applies in the following) is referred to as a first end region Ar1 (FIG. 2 and FIG. 3). Furthermore, on the first holding surface 81, the region that is located on the other end side of the width direction (on the right end side in FIG. 2 and FIG. 3) (separated from the first end region Ar1) and that covers the overall length of the first holding surface 81 is referred to as a second end region Ar2 (FIG. 2 and FIG. 3). Furthermore, on the first holding surface 81, the region that is located at the center of the width direction (located between the first end region Ar1 and the second end region Ar2) and that covers the overall length of the first holding surface 81 is referred to as a first reference position ArC.

Furthermore, the first holding surface 81 is formed as follows.

The first end region Ar1 and the second end region Ar2 are formed by, as illustrated in FIG. 3, a flat surface located on the same plane. The first reference position ArC is set so as to be located on the upper side of the first end region Ar1 and the second end region Ar2. Furthermore, the surface from the first end region Ar1 to the first reference position ArC is connected to a flat inclined plane that is upwardly inclined toward the right side illustrated in FIG. 2 and FIG. 3. Similarly, the surface from the second end region Ar1 to the first reference position ArC is connected to a flat inclined plane that is downwardly inclined toward the right side illustrated in FIG. 2 and FIG. 3.

Namely, the first holding surface 81 has a convex shape.

Then, in the first end region Ar1, as illustrated in FIG. 2 or 3, a first electrode 10 is embedded in the state in which the surface is exposed.

The first electrode 10 generates high frequency energy under the control of the control device 3.

Specifically, the first electrode 10 is formed of, for example, a conductive material, such as copper or aluminum. Furthermore, the first electrode 10 is formed of a substantially rectangular block plate body extending along the central axis of the shaft 6 and disposed such that the upper surface of the first electrode 10 forms the first end region Ar1 on the first holding surface 81. Furthermore, on the first electrode 10, a lead wire (not illustrated) that forms the electric cable C disposed from one end to the other end of the shaft 6.

Furthermore, the first electrode 10 does not need to be the plate body, but the first electrode 10 having a different shape, such as a round bar, having a convex portion that has an interval smaller than that of the first and the second jaws 8 and 9 may also be embedded. Furthermore, the first electrode 10 does not need to be a bulk material, but may also be formed of a conductive thin film, such as platinum, formed by vapor deposition, sputtering, or the like.

Furthermore, the surface of the first electrode 10 does not need to be physically exposed as described above as long as the surface thereof is electrically exposed. Namely, in the state in which the surface is coated with conductive and non-adhesive coating material, such as an Ni-PTFE (polytetrafluoroethylene) film or a conductivity Diamond-Like Carbon (DLC) thin film, even if the surface provides an electric potential as an electrode, this does not depart from the scope of the disclosure.

Configuration of the Second Jaw

The second jaw 9 has a substantially rectangular block shape extending along the central axis of the shaft 6. Examples of a material of the second jaw 9 includes, similarly to the first jaw 8, engineering plastic, such as a PEEK resin; a material with non-conductive and low thermal conductivity, such as a fluorocarbon resin, and a ceramic material with low thermal conductivity, such as alumina and zirconia.

Then, the surface on the lower side of the second jaw 9 illustrated in FIG. 2 and FIG. 3 functions as a second holding surface 91 that holds the biological tissue with the first holding surface 81.

Here, on the second holding surface 91, the region that is located at one end side of the width direction (on the left end side in FIG. 2 and FIG. 3) and that covers the overall length of the second holding surface 91 is referred to as a third end region Ar1′ (FIG. 2 and FIG. 3). Furthermore, on the second holding surface 91, the region that is located at the other end side of the width direction (on the right end side in FIG. 2 and FIG. 3) and that covers the overall length of the second holding surface 91 is referred to as the fourth end region Ar2′. Furthermore, on the second holding surface 91, the region that is located at the center of the width direction (located between the third end region Ar1′ and the fourth end region Ar2′) and that covers the overall length of the second holding surface 91 is referred to as the second reference position ArC′.

Furthermore, as illustrated in FIG. 3, the third end region Ar1′, the fourth end region Ar2′, and the second reference position ArC′ are the regions and the position formed by projecting, in the closed state of the first and the second jaws 8 and 9, the first end region Ar1, the second end region Ar2, the first reference position ArC, respectively, onto the second holding surface 91.

Then, the second holding surface 91 is formed as follows.

The third end region Ar1′ and the fourth end region Ar2′ are formed by, as illustrated in FIG. 3, a flat surface located on the same plane. The second reference position ArC′ is set so as to be located on the upper side of the third end region Ar1′ and the fourth end region Ar2′. Furthermore, the surface from the third end region Ar1′ to the second reference position ArC′ is connected to a flat inclined plane that is upwardly inclined toward the right side illustrated in FIG. 2 and FIG. 3. Similarly, the surface from the fourth end region Ar2′ to the second reference position ArC′ is connected to a flat inclined plane that is downwardly inclined toward the right side illustrated in FIG. 2 and FIG. 3.

Namely, the second holding surface 91 has a concave shape.

Here, as illustrated in FIG. 3, in the closed state of the first and the second jaws 8 and 9, a clearance DE1 between the first and the third end regions Ar1 and Ar1′ is set to be equal to the clearance DE2 between the first and the fourth end regions Ar2 and Ar2′. Furthermore, a clearance DC between the first and the second reference positions ArC and ArC′ is set to be equal to or greater than 1.5 times and equal to or less than 2.5 times the clearances DE1 and DE2.

Then, with the energy treatment instrument 2 according to the first embodiment, the first and the second holding surfaces 81 and 91 are set such that, in the closed state of the first and the second jaws 8 and 9, the clearance between the first and the second holding surfaces 81 and 91 is continuously and smoothly changed (without abrupt change in clearance) from the first end region Ar1 (the third end region Ar1′) and the second end region Ar2 (the second clearance Ar2′) toward the first reference position ArC (the first reference position ArC′) and the clearance DC is the maximum.

Then, in the fourth end region Ar2′, as illustrated in FIG. 2 or FIG. 3, a second electrode 11 is embedded in the state in which the surface is exposed.

The second electrode 11 generates high frequency energy under the control of the control device 3.

Specifically, the second electrode 11 is formed of, for example, a conductive material, such as copper or aluminum. Furthermore, the second electrode 11 is formed of a substantially rectangular block plate body extending along the central axis of the shaft 6 and disposed such that the lower surface of the second electrode 11 forms the fourth end region Ar1′ on the second holding surface 91. Furthermore, a lead wire (not illustrated) that forms the electric cable C disposed from one end side to the other end side of the shaft 6 is joined to the second electrode 11. Then, the first and the second electrodes 10 and 11 can generate high frequency energy due to a supply of high frequency electrical power via the electric cable C (lead wire) performed by the control device 3. Because the electric cable C (lead wire) is connected in order to generate a high frequency electric potential between the first and the second electrodes 10 and 11, by holding biological tissue, it is possible to apply a high frequency current to the biological tissue located between the first and the second electrodes 10 and 11.

Furthermore, similarly to the first electrode 10, the second electrode 11 does not need to be the plate body, but the second electrode 11 having a different shape, such as a round bar, having a convex portion that has an interval smaller than that of the first and the second jaws 8 and 9 may also be embedded. Furthermore, the second electrode 11 does not need to be a bulk material, but may also be formed of a conductive thin film, such as platinum, formed by vapor deposition, sputtering, or the like.

Furthermore, the surface of the second electrode 11 does not need to be physically exposed as described above as long as the surface thereof is electrically exposed. Namely, in the state in which the surface is coated with conductive and non-adhesive coating material, such as an Ni-PTFE film or a conductivity DLC thin film, even if the surface provides an electric potential as an electrode, this does not depart from the scope of the disclosure.

Configuration of the Control Device and the Foot Switch

The foot switch 4 is a part to be operated by the operator's foot. Then, electrical power conduction (supply of high frequency electrical power) from the control device 3 to the energy treatment instrument 2 (the first and the second electrodes 10 and 11) is turned on and off in accordance with the operation of the foot switch 4.

A means for turning on and off the electrical conduction is not limited to the foot switch 4, but another switch, such as a manually operated switch, may also be used.

The control device 3 is formed to include a central processing unit (CPU) or the like and performs overall control of the operation of the energy treatment instrument 2 in accordance with a predetermined control program. More specifically, in accordance with the operation of the foot switch 4 (operation of turning on the electrical power conduction) performed by an operator, the control device 3 supplies, between the first and the second electrodes 10 and 11 via the electric cable C (lead wire), high frequency electrical power at a previously set output rate.

Operation of the Energy Treatment System

In the following, the operation (operation method) of the above described energy treatment system 1 will be described.

FIG. 4 is a diagram illustrating an operation of the energy treatment system 1. Specifically, FIG. 4 is a sectional view associated with FIG. 3 and indicates the state in which biological tissue LT, such as a lumen or a blood vessel, is held by the first and the second jaws 8 and 9.

An operator holds the energy treatment instrument 2 by the operator's hand and inserts the distal end portion (holding portion 7 and a part of the shaft 6) of the energy treatment instrument 2 into the abdominal cavity via the abdominal wall by using, for example, a trocar or the like. Furthermore, the operator operates the operation knob 51 and holds, as illustrated in FIG. 4, the biological tissue LT by the first and the second jaws 8 and 9.

Then, the operator operates the foot switch 4 and turns on the electrical power conduction from the control device 3 to the energy treatment instrument 2. When the electrical power conduction is turned on, the control device 3 supplies high frequency electrical power between the first and the second electrodes 10 and 11 via the electric cable C (lead wire). In response to the supply of the high frequency electrical power, a high frequency current is applied between the first and the second electrodes 10 and 11 and thus Joule heat is generated in the treatment target tissue LT1 of biological tissue LT located between the first and the second electrodes 10 and 11. Then, due to the generated Joule heat, the treatment target tissue LT1 is treated.

With the energy treatment instrument 2 according to the first embodiment described above, the first and the second holding surfaces 81 and 91 are set such that, in the closed state of the first and the second jaws 8 and 9, the clearance between the first and the second holding surfaces 81 and 91 is continuously and smoothly changed (without abrupt change in clearance) from the first end region Ar1 (the third end region Ar1′) and the second end region Ar2 (the fourth end region Ar2′) toward the first reference position ArC (the first reference position ArC′) and the clearance DC is the maximum.

Consequently, by setting the clearance DC of the center position of the first and the second holding surfaces 81 and 91 to the maximum, it is possible to design a variation in current density of the high frequency current applied between the first and the second electrodes 10 and 11. Namely, the current density is high around the end portion on the inner side of the width direction of the first electrode 10 (on the first reference position ArC side) and around the end portion on the inner side of the width direction of the second electrode 11 (on the second reference position ArC′ side) and the current density is low between the first and the second reference positions ArC and ArC′. Thus, in accordance with the current density of the high frequency current, in the treatment target tissue LT1, the similar variation is also generated in the heat-generating density. Namely, the heat-generating density is high at the tissue around the end portion on the inner side of the width direction of the first electrode 10 and the tissue around the end portion on the inner side of the width direction of the second electrode 11 and the heat-generating density is low at the tissue between the first and the second reference positions ArC and ArC′.

Accordingly, by limiting a heat transfer path in the treatment target tissue LT1 and by lowering the heat-generating density of the tissue between the first and the second reference positions ArC and ArC′ in which temperature is likely to be increased, the speed of rise in temperature of the tissue may be reduced. In contrast, in the treatment target tissue LT1, the heat-generating density of the tissue around the end portion on the inner side of the width direction of the first electrode 10 and the tissue around the end portion on the inner side of the width direction of the second electrode 11 is higher than that of the tissue between the first and the second reference positions ArC and ArC′. However, the unprocessed biological tissue LT with a large heat capacity is located in a close vicinity of these pieces of tissue and the heat transfer path has been secured. Consequently, the speed of rise in temperature of these pieces of tissue may be associated with the speed of rise in temperature of the tissue located between the first and the second reference positions ArC and ArC′.

Furthermore, because the clearance between the first and the second holding surfaces 81 and 91 is continuously and smoothly changed, the heat-generating density in the treatment target tissue LT1 is also continuously and smoothly changed. By considering this state so as to cancel out the heat transfer level, it is possible to simultaneously and uniformly raise the temperature in a wide area.

Based on the above, with the energy treatment instrument 2 according to the first embodiment, an advantage is provided in that it is possible to simultaneously and uniformly raise the temperature in a large area of the treatment target tissue LT1 in the biological tissue LT and appropriately perform treatment on the large area of the treatment target tissue LT1.

Furthermore, in the energy treatment instrument 2 according to the first embodiment, the first holding surface 81 has a convex shape. Consequently, when joining the biological tissue LT, such as a lumen or a blood vessel, by holding the biological tissue LT by the first and the second holding surfaces 81 and 91, it is possible to efficiently push the contents in a lumen or a blood vessel out of, at least, the region LT1 that is the treatment target through the first and the second jaws 8 and 9 by the first holding surface 81 having the convex shape. Namely, the content unnecessary for the joining may be removed, thereby stably joining the biological tissue LT.

### Modification of the First Embodiment

In the first embodiment described above, the second electrode 11 is disposed in the fourth end region Ar2′; however, the embodiment is not limited to this, but the second electrode 11 may also be disposed in the second end region Ar2.

Furthermore, similarly, the first electrode 10 is disposed in the first end region Ar1; however, the embodiment is not limited to this, but the first electrode 10 may also be disposed in the third end region Ar1′.

Although the path for the high frequency current is changed, this does not strongly affect the distribution of the heat-generating density as a whole.

### Second Embodiment

In the following, a second embodiment will be described.

In a description of the second embodiment, the same components as those of the first embodiment described above are denoted by the same reference numerals, and detailed explanation thereof will be omitted or simplified.

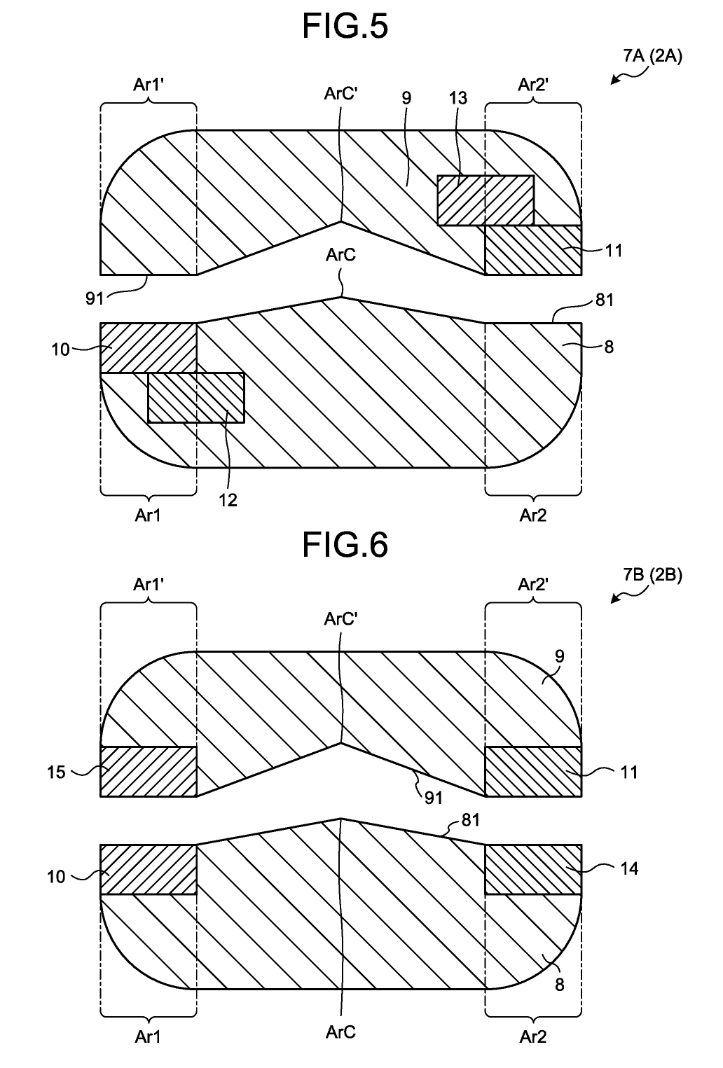

FIG. 5 is a diagram illustrating a holding portion 7A of an energy treatment instrument 2A according to a second embodiment. Specifically, FIG. 5 is a sectional view associated with FIG. 3.

In the energy treatment instrument 2 according to the second embodiment, as illustrated in FIG. 5, a first and a second cooling members 12 and 13 are added to the energy treatment instrument 2 (FIG. 3) described in the first embodiment.

Specifically, the first cooling member 12 has a function as a cooling member, is thermally in contact with at least the first electrode 10, and cools the first electrode 10.

In the first embodiment, the first cooling member 12 has a configuration in which a latent heat storage material is sealed inside the first cooling member 12. Then, as illustrated in FIG. 5, the first cooling member 12 is provided inside the jaw in which the first electrode 10 is provided, i.e., in this case, the first jaw 8, and is disposed so as to be in contact with the surface of the first electrode 10 at the opposite side from the surface on which the first holding surface 81 is brought into contact.

Here, the latent heat storage material described above is the substance that exhibits the same thermal behavior as that exhibited by other substances up to a certain temperature, but that exhibits a phase transition specific for the substance at a certain temperature, and that can hold, by using a heat absorbing action due to latent heat caused by the phase transition, a large amount of heat per unit volume when compared with the other substances. Examples of a material of the latent heat storage material include a solid substance at a room temperature, such as paraffin, polylactic acid, magnesium hydroxide, erythritol, and mannitol. For example, the operating temperature of paraffin (temperature at which a phase transition from a solid to liquid occurs) is about 40° C. Furthermore, the operating temperature of erythritol is about 120° C. Namely, the material of the latent heat storage material may be selected based on the desired operating temperature of heat absorption.

The second cooling member 13 has a function as a cooling member, is thermally in contact with at least the second electrode 11, and cools the second electrode 11.

Furthermore, the second cooling member 13 may also have the same configuration as that of the first cooling member 12. Then, the second cooling member 13 is provided inside the jaw in which the second electrode 11 is provided, i.e., in this case, the second jaw 9, and is disposed so as to be in contact with the surface of the second electrode 11 at the opposite side from the surface on which the second holding surface 91 is brought into contact.

The energy treatment instrument 2A according to the second embodiment described above provides, in addition to the same effect as that obtained in the first embodiment described above, the following advantages.

When applying energy to the biological tissue LT, due to the heat generated in the treatment target tissue LT1 (in particular, the tissue in the vicinity of the first and the second electrodes 10 and 11), the temperature of the first and the second electrodes 10 and 11 also rises due to the heat transfer from the treatment target tissue LT1. Then, the thermal conductivity of the first and the second electrodes 10 and 11 is higher than that of the biological tissue LT. Consequently, the heat transferred to the first and the second electrodes 10 and 11 is conducted inside the first and the second electrodes 10 and 11 faster than the heat conducted in the biological tissue LT in the width direction. Based on this, because the entirety of the first and the second electrodes 10 and 11 is heated, in the biological tissue LT, heat is transferred from the portion that is in contact with the first and the second electrodes 10 and 11 to a peripheral tissue that is located on the outer side of the width direction of the first and the second jaws 8 and 9 and that is located around the treatment target tissue LT1. Namely, if the temperature of the first and the second electrodes 10 and 11 significantly exceeds the temperature of thermal denaturation of protein of the peripheral tissue, the effect of heat exerted to the peripheral tissue is not able to be ignored.

With the energy treatment instrument 2A according to the second embodiment, in the first jaw 8, the first electrode 10 is cooled by the first cooling member 12. Furthermore, in the second jaw 9, the second electrode 11 is cooled by the second cooling member 13. Consequently, by cooling the heat of the first and the second electrodes 10 and 11 by the first and the second cooling members 12 and 13, it is possible to reduce the thermal effect exerted on the peripheral tissue and avoid a decrease in natural healing power of the peripheral tissue.

Furthermore, in the energy treatment instrument 2A according to the second embodiment, the first and the second cooling members 12 and 13 are not positive cooling means for compulsory refluxing a fluid, such as liquid or gas, but cooling the first and the second electrodes 10 and 11 by using a latent heat storage material. In a method of compulsory reflux, because the first and the second electrodes 10 and 11 are thermally maintained by the temperature of refrigerant, there is a need to consider the possibility of long treatment hours due to supercooling, an increase in needed electrical power, and unexpected poor treatment due to rapid temperature change. On that point, when using a latent heat storage material, up to the operating temperature of the latent heat storage material, a rise in temperature of the treatment target tissue LT1 is not greatly prevented, thereby continuing the treatment. In contrast, if the temperature of the latent heat storage material becomes equal to or greater than the operating temperature, heat absorption is started based on a nonlinear behavior of the latent heat storage material and thus it is possible to prevent the temperature from rising above. Thus, it is possible to perform treatment on the treatment target tissue LT1 without greatly deviating from the target or increasing the treatment period of time and electrical power.

### Modification of the Second Embodiment

In the second embodiment described above, the first and the second cooling members 12 and 13 is configured so as to be in contact with the outer surface of the first and the second electrodes 10 and 11; however, the configuration is not limited to this. For example, it may also be possible to use the configuration such that the first and the second electrodes 10 and 11 are formed by using hollow members, the latent heat storage materials described above are disposed inside the first and the second electrodes 10 and 11, and the first and the second electrodes 10 and 11 are cooled.

Furthermore, in the second embodiment described above, each of the first and the second cooling members 12 and 13 uses the latent heat storage material formed of a solid substance at a room temperature including a material that is sealed in the form of a capsule and that is visually solid; however, the configuration is not limited to this, but it may also be possible to use a heat pipe using a latent heat storage material formed of a liquid substance at a room temperature, such as water, a CFC substitutes.

Furthermore, the first and the second cooling members 12 and 13 are preferably formed of latent heat storage materials; however, the configuration is not limited to this. It may also be possible to use the configuration by providing a coolant line so as to be in contact with inside or an outer surface of the first and the second electrodes 10 and 11 and by allowing a cooling medium, such as water, oil, nitrogen, or carbon dioxide, to flow through the coolant line.

In the second embodiment described above, the first and the second cooling members 12 and 13 are provided at the first and the second jaws 8 and 9, respectively; however, the configuration is not limited to this. According to the configuration, because it is possible to reduce the effect of the heat exerted on the other area of treatment and increase the treatment area, one of the first and the second cooling members 12 and 13 may also be omitted.

### Third Embodiment

In the following, a third embodiment will be described.

In a description of the third embodiment, the same components as those of the first embodiment described above are denoted by the same reference numerals, and detailed explanation thereof will be omitted or simplified.

FIG. 6 is a diagram illustrating a holding portion 7B of an energy treatment instrument 2B according to a third embodiment. Specifically, FIG. 6 is a sectional view associated with FIG. 3.

In the energy treatment instrument 2B according to the third embodiment, as illustrated in FIG. 6, a third and a fourth electrodes 14 and 15 are added to the energy treatment instrument 2 (FIG. 3) described in the first embodiment.

Specifically, as illustrated in FIG. 6, the third electrode 14 is embedded in the second end region Ar2 in the state in which the surface is exposed and generates high frequency energy under the control of the control device 3. The third electrode 14 is formed of a conductive material, such as copper or aluminum. Furthermore, the third electrode 14 is formed of a substantially rectangular block plate body extending along the central axis of the shaft 6 (the same thickness size as that of the first electrode 10) and disposed such that the upper surface forms the second end region Ar2 on the first holding surface 81. Furthermore, a lead wire (not illustrated) that forms the electric cable C disposed from one end side to the other end side of the shaft 6 is joined to the third electrode 14.

Furthermore, as illustrated in FIG. 6, the fourth electrode 15 is embedded in the third end region Ar1′ in the state in which the surface is exposed and then generates high frequency energy under the control of the control device 3. The fourth electrode 15 is formed of a conductive material, such as copper or aluminum. Furthermore, the fourth electrode 15 is formed of a substantially rectangular block plate body extending along the central axis of the shaft 6 (the same thickness size as that of the second electrode 11) and disposed such that the lower surface forms the third end region Ar1′ on the second holding surface 91. Furthermore, lead wire (not illustrated) that forms the electric cable C disposed from one end side to the other end side of the shaft 6 is joined to the fourth electrode 15. Then, each of the first to the fourth electrodes 10, 11, 14, and 15 generates high frequency energy because high frequency electrical power is supplied via the electric cable C (lead wire) by the control device 3 (flowing high frequency current into the treatment target tissue LT1). Furthermore, when the high frequency electrical power is being supplied, the first and the fourth electrodes 10 and 15 are at the same electric potential, whereas the second and the third electrodes 11 and 14 are at the other same electric potential. Furthermore, the phase of the high frequency electrical power of the first and the fourth electrodes 10 and 15 is different from that of the second and the third electrodes 11 and 14 by 180 degrees. Namely, the high frequency current flows, in the width direction of the first and the second jaws 8 and 9, between the first and the fourth electrodes 10 and 15 and the second and the third electrodes 11 and 14.

Furthermore, each of the third and the fourth electrodes 14 and 15 does not need to be the plate body, but each of the third and the fourth electrodes 14 and 15 having a different shape, such as a round bar, having a convex portion that has an interval smaller than that of the first and the second jaws 8 and 9 may also be embedded. Furthermore, each of the third and the fourth electrodes 14 and 15 does not need to be a bulk material, but may also be formed of a conductive thin film, such as platinum, formed by vapor deposition, sputtering, or the like.

Furthermore, the surface of each of the third and the fourth electrodes 14 and 15 does not need to be physically exposed as described above as long as the surface thereof is electrically exposed. Namely, in the state in which the surface is coated with conductive and non-adhesive coating material, such as an Ni-PTFE film or a conductivity DLC thin film, even if the surface provides an electric potential as an electrode, this does not depart from the scope of the disclosure.

The energy treatment instrument 2B according to the third embodiment described above provides, in addition to the same advantages as those described in the first embodiment described above, the following advantages.

In the energy treatment instrument 2B according to the third embodiment, the third electrode 14 is disposed in the second end region Ar2 and the fourth electrode 15 is disposed in the third end region Ar1′. Consequently, it is possible to reduce a difference in temperature between the tissue around the first end region Ar1 and the tissue around the third end region Ar1′ and a difference in temperature between the tissue around the second end region Ar2 and the tissue around the fourth end region Ar2′ and it is possible to further uniformly raise the temperature of the treatment target tissue LT1. Furthermore, because the contact area of the biological tissue LT is doubled and, regarding the high frequency energy needed for treatment, the current density for each electrode may be halved, it is also possible to reduce the current needed for the device.

### Modification of the Third Embodiment

In the third embodiment described above, high frequency electrical power may also be simultaneously supplied between the first and the second electrodes 10 and 11 and between the third and the fourth electrodes 14 and 15, or alternatively, high frequency electrical power may also be alternately and time divisionally supplied between the first and the second electrodes 10 and 11 and between the third and the fourth electrodes 14 and 15 (for example, at an interval of 0.1 seconds).

Furthermore, in the third embodiment described above, high frequency electrical power may also be simultaneously supplied between the first and the third electrodes 10 and 14 and between the second and the fourth electrodes 11 and 15, or alternatively, high frequency electrical power may also be alternately and time divisionally supplied between the first and the third electrodes 10 and 14 and between the second and the fourth electrodes 11 and 15 (for example, at an interval of 0.1 seconds).

Incidentally, in a case of using the above described configuration in which “high frequency electrical power is simultaneously supplied”, there is a possibility that the temperature of all of the first to the fourth electrodes 10, 11, 14, and 15 becomes high due to the heat transferred from the treatment target tissue LT1 (in particular, the tissue in the vicinity of the first to the fourth electrodes 10, 11, 14, and 15). Namely, due to the heat transferred from the first to the fourth electrodes 10, 11, 14, and 15, in the biological tissue LT, the effect of heat exerted to the peripheral tissue that is located on the outer side of the width direction of the first and the second jaws 8 and 9 and that is located around the treatment target tissue LT1 is not possibly be ignored.

However, in a case of using the above described configuration in which “high frequency electrical power is alternately and time divisionally supplied”, because high frequency electrical power is not continuously supplied to the same electrode, it is possible to relatively reduce a temperature rise in the first to the fourth electrodes 10, 11, 14, and 15. Namely, it is possible to further reduce the effect of heat exerted to the peripheral tissue.

### Fourth Embodiment

In the following, a fourth embodiment will be described.

In a description of the fourth embodiment, the same components as those of the third embodiment described above are denoted by the same reference numerals, and detailed explanation thereof will be omitted or simplified.

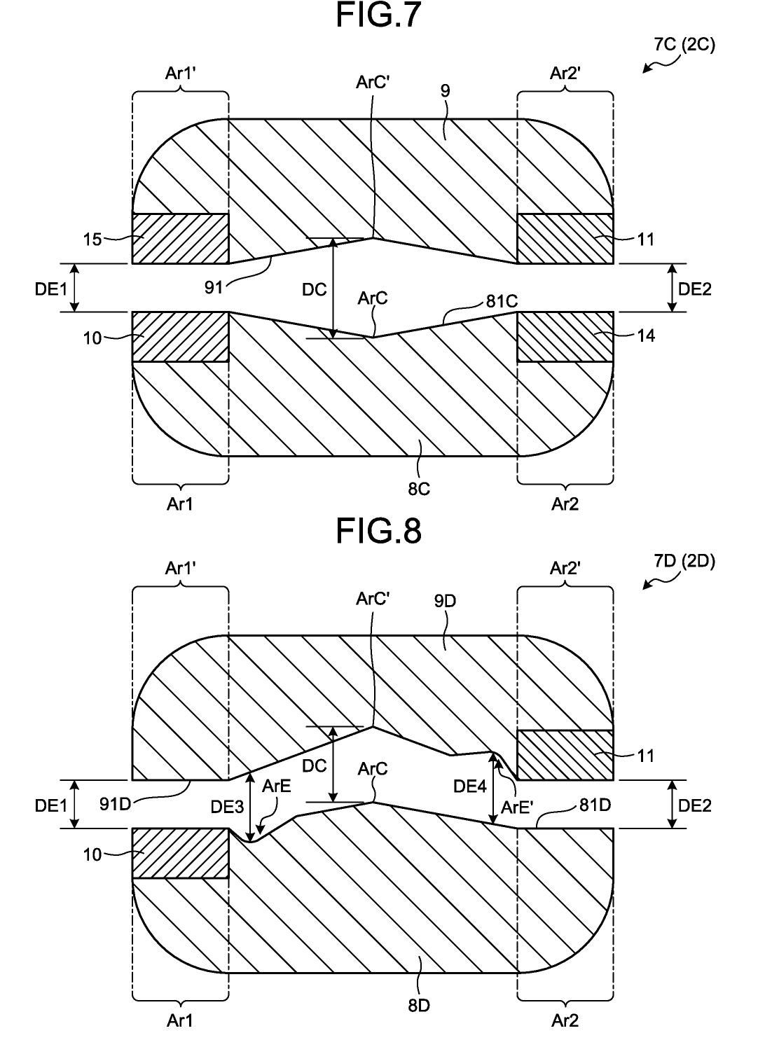

FIG. 7 is a diagram illustrating a holding portion 7C of an energy treatment instrument 2C according to a fourth embodiment. Specifically, FIG. 7 is a sectional view associated with FIG. 6.

In the energy treatment instrument 2C according to the fourth embodiment, as illustrated in FIG. 7, when compared with the energy treatment instrument 2B (FIG. 6) according to the third embodiment described above, a first jaw 8C having a first holding surface 81C that has a shape different from that of the first holding surface 81 is used instead of the first jaw 8.

Specifically, the first holding surface 81C is formed as follows.

The first end region Ar1 (the upper surface of the first electrode 10) and the second end region Ar2 (the upper surface of the third electrode 14) are formed by, as illustrated in FIG. 7, a flat surface located on the same plane. The first reference position ArC is set so as to be located on the lower side of the first end region Ar1 and the second end region Ar2. Furthermore, the surface from the first end region Ar1 to the first reference position ArC is connected to a flat inclined plane that is downwardly inclined toward the right side illustrated in FIG. 7. Similarly, the surface from the second end region Ar2 to the first reference position ArC is connected to a flat inclined plane that is upwardly inclined toward the right side illustrated in FIG. 7.

Namely, the first holding surface 81C has a concave shape.

Here, in the energy treatment instrument 2C according to the fourth embodiment, similarly to the first embodiment described above, the clearance DE1 between the first and the third end regions Ar1 and Ar1′ is set so as to be the same clearance as the clearance DE2 between the first and the fourth end regions Ar2 and Ar2′. Furthermore, the clearance DC between the first and the second reference positions ArC and ArC′ is set to be greater than 1.5 times and less than 2.5 times the clearances DE1 and DE2.

Then, in the energy treatment instrument 2C according to the fourth embodiment, similarly to the first embodiment, the first and the second holding surfaces 81C and 91 are set such that, in the closed state of the first and the second jaws 8C and 9, the clearance between the first and the second holding surfaces 81C and 91 is continuously and smoothly changed (without abrupt change in clearance) from the first end region Ar1 (the third end region Ar1′) and the second end region Ar2 (the fourth end region Ar2′) toward the first reference position ArC (the second reference position ArC′) and the clearance DC is the maximum.

The energy treatment instrument 2C according to the fourth embodiment described above provides the same advantages as those described above in the third embodiment.

### Modification of the Fourth Embodiment

In the fourth embodiment, four electrodes, i.e., the first to the fourth electrodes 10, 11, 14, and 15, are provided; however, the configuration is not limited to this. Similarly to the first embodiment described above, it may also possible to use the configuration in which only the two electrodes of the first and the second electrodes 10 and 11 are provided or, alternatively, only the two electrodes of the first and the third electrodes 10 and 14 or the second and the fourth electrodes 11 and 13 are provided.

Furthermore, in the fourth embodiment described above, one of the first and the second holding surfaces 81C and 91 may also be formed by a flat surface.

### Fifth Embodiment

In the following, a fifth embodiment will be described.

In a description of the fifth embodiment, the same components as those of the first embodiment described above are denoted by the same reference numerals, and detailed explanation thereof will be omitted or simplified.

FIG. 8 is a diagram illustrating a holding portion 7D of an energy treatment instrument 2D according to a fifth embodiment. Specifically, FIG. 8 is a sectional view associated with FIG. 3.

In the energy treatment instrument 2D according to the fifth embodiment, as illustrated in FIG. 8, when compared with the energy treatment instrument 2 (FIG. 3) described above in the first embodiment, a first jaw 8D having a first holding surface 81D that has the shape different from that of the first holding surface 81 is used instead of the first jaw 8 and a second jaw 9D having a second holding surface 91D that has the shape different from that of the second holding surface 91 is used instead of the second jaw 9.

Here, on the first holding surface 81D, the position that is located on the first end region Ar1 side between the first end region Ar1 and the first reference position ArC and that covers the overall length of the first holding surface 81D is referred to as a first auxiliary position ArE.

Furthermore, the first holding surface 81D has, when compared with the first holding surface 81 according to the first embodiment described above, a concave curved surface shape in which the surface from the first end region Ar1 to the first auxiliary position ArE is downwardly depressed.

Furthermore, on the second holding surface 91D, the position that is located on the fourth end region Ar2′ side between the fourth end region Ar2′ and the second reference position ArC′ and that covers the overall length of the second holding surface 91D is referred to as a second auxiliary position ArE′.

Furthermore, the second holding surface 91D has, when compared with the second holding surface 91 according to the first embodiment described above, a concave curved surface shape in which the surface from the fourth end region Ar2′ to the second auxiliary position ArE′ upwardly recessed.

Here, in the energy treatment instrument 2D according to the fifth embodiment, in the closed state of the first and the second jaws 8D and 9D, a clearance DE3 (FIG. 8) between the first and the second holding surfaces 81D and 91D at the first auxiliary position ArE is set to be the same clearance as a clearance DE4 (FIG. 8) between the first and the second holding surfaces 81D and 91D at the second auxiliary position ArE′. Furthermore, the clearance DE3 (DE4) is set to be greater than the clearance DE1 between the first and the third end regions Ar1 and Ar1′ (the clearance DE2 between the first and the second clearances Ar2 and Ar2′) and is set to be equal to or less than the clearance DC between the first and the second reference positions ArC and ArC′.

Then, in the energy treatment instrument 2D according to the fifth embodiment, similarly to the first embodiment, the first and the second holding surfaces 81D and 91D are set such that, in the closed state of the first and the second jaws 8D and 9D, the clearance between the first and the second holding surfaces 81D and 91D is continuously and smoothly changed (without abrupt change in clearance) from the first end region Ar1 (the third end region Ar1′) and the second end region Ar2 (the fourth end region Ar2′) toward the first reference position ArC (the second reference position ArC′) and the clearance DC is the maximum.

The energy treatment instrument 2D according to the fifth embodiment described above provides, in addition to the same effect as that obtained in the first embodiment described above, the following advantages.

When providing only the two electrodes of the first and the second electrodes 10 and 11 and applying a high frequency current between the first and the second electrodes 10 and 11, the current density around the end portion on the inner side of the width direction (on the first reference position ArC side) of the first electrode 10 and around the end portion on the inner side of the width direction (on the second reference position ArC′ side) on the second electrode 11 tends to be high.

In the energy treatment instrument 2D according to the fifth embodiment, in the first and the second holding surfaces 81D and 91D, the first and the second auxiliary positions ArE and ArE′ located in the vicinity of the first and the second electrodes 10 and 11 are formed in a concave curved surface shape. Then, the clearance DE3 between the first and the second holding surfaces 81D and 91D at the first auxiliary position ArE (the clearance DE4 between the first and the second holding surfaces 81D and 91D at the second auxiliary position ArE′) is set to be greater than the clearance DE1 between the first and the third end regions Ar1 and Ar1′ (the clearance DE2 between the first and the second clearances Ar2 and Ar2′) and is set to be equal to or less than the clearance DC between the first and the second reference positions ArC and ArC′.

Consequently, it is possible to reduce the current density around end portion on the inner side of the width direction of the first electrode 10 and around the end portion on the inner side of the width direction of the second electrode 11 and also consequently reduce the heat-generating density. Thus, it is possible to more uniformly raise the temperature of the treatment target tissue LT1.

### Sixth Embodiment

In the following, a sixth embodiment will be described.

In a description of the sixth embodiment, the same components as those of the fifth embodiment described above are denoted by the same reference numerals, and detailed explanation thereof will be omitted or simplified.

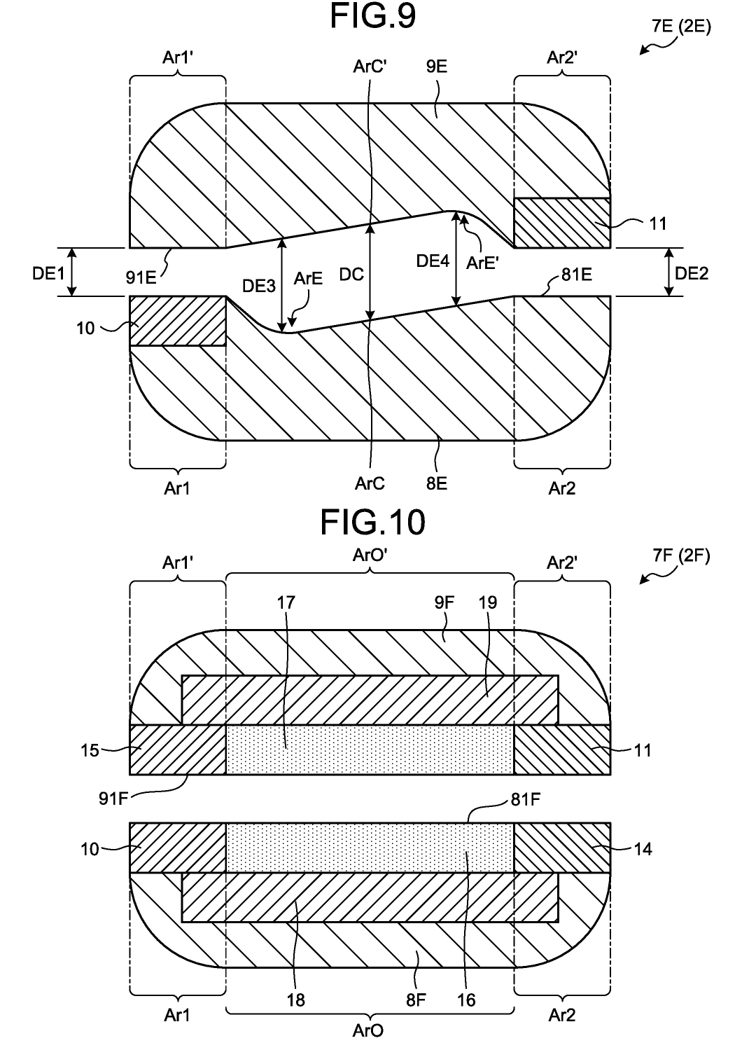

FIG. 9 is a diagram illustrating a holding portion 7E of an energy treatment instrument 2E according to a sixth embodiment. Specifically, FIG. 9 is a sectional view associated with FIG. 8.

In the energy treatment instrument 2E according to the sixth embodiment, as illustrated in FIG. 9, when compared with the energy treatment instrument 2D (FIG. 8) according to the fifth embodiment described above, a first jaw 8E having a first holding surface 81E that has a shape different from that of the first holding surface 81D is used instead of the first jaw 8D and a second jaw 9E having a second holding surface 91E that has a shape different from that of the second holding surface 91D is used instead of the second jaw 9D.

Specifically, as illustrated in FIG. 9, when compared with the first and the second holding surfaces 81D and 91D according to the fifth embodiment described above, in the closed state of the first and the second jaws 8E and 9E, on the first and the second holding surfaces 81E and 91E, the clearance between the first and the second holding surfaces 81E and 91E is set to be the same in the region from the first auxiliary position ArE to the second auxiliary position ArE′. Consequently, the clearance DE3 between the first and the second holding surfaces 81E and 91E at the first auxiliary position ArE, the clearance DC between the first and the second reference positions ArC and ArC′, and the clearance DE4 between the first and the second holding surfaces 81E and 91E at the second auxiliary position ArE′ are the same.

Namely, in the energy treatment instrument 2E according to the sixth embodiment, similarly to the fifth embodiment, the first and the second holding surfaces 81E and 91E are set such that, in the closed state of the first and the second jaws 8E and 9E, the clearance between the first and the second holding surfaces 81E and 91E is continuously and smoothly changed (without abrupt change in clearance) from the first end region Ar1 (the third end region Ar1′) and the second end region Ar2 (the fourth end region Ar2′) toward the first reference position ArC (the second reference position ArC′) and the clearances (DE3, DC, and DE4) are the maximum at the first auxiliary position ArE, the first and the second reference positions ArC and ArC′, and the second auxiliary position ArE′.

The energy treatment instrument 2E according to the sixth embodiment described above provides the same advantages as those provided in the fifth embodiment described above.

As described above in the fifth embodiment, when providing only the two electrodes of the first and the second electrodes 10 and 11 and applying a high frequency current between the first and the second electrodes 10 and 11, the current density around the end portion on the inner side of the width direction (on the first reference position ArC side) of the first electrode 10 and the end portion around the inner side of the width direction (on the second reference position ArC′ side) of the second electrode 11 tends to be high. Namely, the portion of the highest heat-generating density in the treatment target tissue LT1 may possibly be the tissue other than the tissue between the first and the second reference positions ArC and ArC′.

In such a case, it may also be possible to use a configuration such that the clearance between the first and the second holding surfaces 81E and 91E is the maximum at the position other than the first and the second reference positions ArC and ArC′ (in the sixth embodiment, the first and the second auxiliary positions ArE and ArE′). Namely, the first and the second reference positions are not limited to the first and the second reference positions ArC and ArC′, respectively, that are located at the center of the width direction, but may also use the positions deviated from the center of the width direction as the first and the second reference positions.

### Seventh Embodiment

In the following, a seventh embodiment will be described.

In a description of the seventh embodiment, the same components as those of the third embodiment described above are denoted by the same reference numerals, and detailed explanation thereof will be omitted or simplified.

FIG. 10 is a diagram illustrating a holding portion 7F of an energy treatment instrument 2F according to a seventh embodiment. Specifically, FIG. 10 is a sectional view associated with FIG. 6.

In the energy treatment instrument 2F according to the seventh embodiment, as illustrated in FIG. 10, when compared with the energy treatment instrument 2B (FIG. 6) according to the third embodiment described above, a first jaw 8F having a first holding surface 81F that has a shape different from that of the first holding surface 81 is used instead of the first jaw 8, a second jaw 9F having a second holding surface 91F that has a shape different from that of the second holding surface 91 is used instead of the second jaw 9 and, furthermore, a first and a second thermal resistance members 16 and 17 and a first and a second cooling members 18 and 19 are added.

Here, on the first holding surface 81F, the region that is located between the first end region Ar1 and the second end region Ar2, that is in contact with the first end region Ar1 and the second end region Ar2, and that covers the overall length of the first holding surface 81F is referred to as a first central region Ar0.

Then, the first central region Ar0 is formed by a flat surface so as to be flush with the first end region Ar1 and the second end region Ar2.

Furthermore, on the second holding surface 91F, the region that is located between the third end region Ar1′ and the fourth end region Ar2′, that is in contact with the third end region Ar1′ and the fourth end region Ar2′, and that covers the overall length of the second holding surface 91F is referred to as a second central region Ar0′.

Then, the second central region ArO′ is formed by a flat surface such that the second central region ArO′ is flush with the third end region Ar1′ and the fourth end region Ar2′.

Furthermore, as illustrated in FIG. 10, the second central region ArO′ is the region formed by projecting the first central region ArO onto the second holding surface 91F in the closed state of the first and the second jaws 8F and 9F.

Namely, in the energy treatment instrument 2F according to the seventh embodiment, in the closed state of the first and the second jaws 8F and 9F, the clearance between the first and the second holding surfaces 81F and 91F is set to be the same at any position on the first and the second holding surfaces 81F and 91F.

The first thermal resistance member 16 embedded in the first central region ArO in the state in which, as illustrated in FIG. 10, the surface is exposed.

Specifically, the first thermal resistance member 16 is formed of a member with low thermal conductivity that is lower than that of the first and the third electrodes 10 and 14. Furthermore, the first thermal resistance member 16 is formed of a substantially rectangular block plate body that extends along the central axis of the shaft 6 and that has the same thickness size as that of the first and the third electrodes 10 and 14 and is disposed such that the upper surface of the first thermal resistance member 16 is flush with each of the upper surfaces of the first and the third electrodes 10 and 14 and forms the first central region ArO on the first holding surface 81F.

Any material may be used for a material of the first thermal resistance member 16 as long as the material with thermal conductivity that is lower than that of the first and the third electrodes 10 and 14 and examples of a material of the first thermal resistance member 16 include a metal with low thermal conductivity, such as titanium; a low density metal formed of a porous body; a resin, such as tetrafluoroethylene perfluoroalkoxy ethylene copolymer (PFA) or PTFE; a hollow resin; porous thermosetting plastics; a ceramic with low thermal conductivity, such as alumina, zirconia, macerite; and a porous ceramic.

Namely, in the seventh embodiment, by setting the thermal conductivity of the first central region ArO (the first thermal resistance member 16) to the thermal conductivity lower than that of the first end region Ar1 (the first electrode 10) and the second end region Ar1 (the third electrode 14), the thermal resistance against the biological tissue LT in the first central region ArO (the first thermal resistance member 16) is set to be higher than the thermal resistance against the biological tissue LT in the first end region Ar1 (the first electrode 10) and the second end region Ar2 (the third electrode 14).

Furthermore, as illustrated in FIG. 10, the second thermal resistance member 17 is embedded in the second central region ArO′ in the state in which the surface of the second thermal resistance member 17 is exposed.

Furthermore, the second thermal resistance member 17 may also have the same configuration as that of the first thermal resistance member 16. Then, the second thermal resistance member 17 is disposed such that the lower surface of the second thermal resistance member 17 is flush with each of the lower surfaces of the second and the fourth electrodes 11 and 15 and forms the second central region ArO′ on the second holding surface 91F.

Namely, in also the second holding surface 91F, similarly to the first holding surface 81F, by setting the thermal conductivity of the second central region ArO′ (the second thermal resistance member 17) to the thermal conductivity lower than that of the third end region Ar1′ (the fourth electrode 15) and the fourth end region Ar2′ (the second electrode 11), the thermal resistance against the biological tissue LT in the second central region ArO′ (the second thermal resistance member 17) is set to be higher than the thermal resistance against the biological tissue LT in the third end region Ar1′ (the fourth electrode 15) and the fourth end region Ar1′ (the second electrode 11).

The first cooling member 18 is thermally in contact with the first and the third electrodes 10 and 14 and cools the first and the third electrodes 10 and 14. Then, the first cooling member 18 is provided inside the first jaw 8F and is disposed so as to be in contact with each of the lower surfaces of the first and the third electrodes 10 and 14 and the first thermal resistance member 16.

The second cooling member 19 is thermally in contact with the second and the fourth electrodes 11 and 15 and cools the second and the fourth electrodes 11 and 15. Then, the second cooling member 19 is provided inside the second jaw 9F and is disposed so as to be in contact with each of the upper surfaces of the second and the fourth electrodes 11 and 15 and the second thermal resistance member 17.

Furthermore, the first and the second cooling members 18 and 19 may also have the same configuration as that of the first and the second cooling members 12 and 13 according to the second embodiment described above.

The seventh embodiment described above provides, in addition to the same advantages as those described in the second embodiment, the following advantages.

In the energy treatment instrument 2F according to the seventh embodiment, the first thermal resistance member 16 (the second thermal resistance member 17) forms the first central region ArO (the second central region ArO′) on the first holding surface 81F (the second holding surface 91F). Furthermore, the first thermal resistance member 16 (the second thermal resistance member 17) has thermal conductivity that is lower than that of the first and the third electrodes 10 and 14 (the second and the fourth electrodes 11 and 15) and the thermal resistance against the biological tissue LT is higher than that of the first and the third electrodes 10 and 14 (the second and the fourth electrodes 11 and 15). Consequently, it is possible to reduce an amount of heat absorbed from the biological tissue LT by the first and the second thermal resistance members 16 and 17 than an amount of heat absorbed from biological tissue LT by the first to the fourth electrodes 10, 11, 14, and 15, thereby preventing a decrease in temperature of the treatment target tissue LT1.

### Modification of the Seventh Embodiment

In the seventh embodiment described above, by providing the first and the second thermal resistance members 16 and 17 formed of a material having low thermal conductivity, the thermal resistance of the first and the second central regions ArO and ArO′ against the biological tissue LT is set to be higher than that of the first and the third end regions Ar1 and Ar1′ (the first and the fourth electrodes 10 and 15) and the first and the fourth end regions Ar2 and Ar2′ (the second and the third electrodes 11 and 14); however, the configuration is not limited to this.

For example, by omitting the first and the second thermal resistance members 16 and 17 and performing surface treatment (for example, etching, sandblasting, etc.) in order to roughen the surface of the first and the second central regions ArO and ArO′ on the first and the second holding surfaces 81F and 91F or originally forming to be a flat-joint or mesh surface. Namely, by roughening the roughness of the surface in the first and the second central regions ArO and ArO′, the thermal resistance against the biological tissue LT in the first and the second central regions ArO and ArO′ is set to be higher than that of the first and the third end regions Ar1 and Ar1′ and the first and the fourth end regions Ar2 and Ar2′.

Furthermore, for example, the first and the second central regions ArO and ArO′ on the first and the second holding surfaces 81F and 91F is formed by a concave portion by omitting the first and the second thermal resistance members 16 and 17. Namely, by using air space in the concave portion, thermal resistance against the biological tissue LT in the first and the second central regions ArO and ArO′ is set to be higher than that of the first and the third end regions Ar1 and Ar1′ and the first and the fourth end regions Ar2 and Ar2′.

In the seventh embodiment described above, the first and the second thermal resistance members 16 and 17 are provided in the first and the second jaws 8F and 9F, respectively; however, the configuration is not limited to this and one of the first and the second thermal resistance members 16 and 17 may also be omitted. Similarly, one of the first and the second cooling members 18 and 19 may also be omitted.

### OTHER EMBODIMENTS

The embodiments for carrying out the present disclosure have been described above; however, the present disclosure is not limited only by the first to seventh embodiments and modifications thereof.

In the first to the sixth embodiments and the modifications thereof described above, the first holding surface is not limited to the first holding surface 81 (81C to 81E) described in the first to the sixth embodiments and the modifications thereof described above, but may also be formed of another surface as long as the surface is continuous and smooth surface without a sharp turn. The same applies to the second holding surface 91 (91D and 91E).

In the first to the sixth embodiments and the modifications thereof described above, It may also be possible to use the configuration in which the first jaw 8 (8C to 8E) and the second jaw 9 (9D and 9E) is made of the same material as that used for, for example, the first electrode 10 and, on the first holding surface 81 (81C to 81E) and the second holding surface 91 (91D and 91E), a coating material, such as PTFE or a silicon, is added to the region excluding the first to the fourth electrodes 10, 11, 14, and 15.

In the first to the seventh embodiments and the modifications thereof, the energy treatment instrument 2 (2A to 2F) treats the biological tissue LT by applying only high frequency energy; however, the configuration is not limited to this. In addition to high frequency energy, at least one of optical energy, such as ultrasonic energy or laser, and thermal energy is applied to the biological tissue LT for the treatment.

According to an aspect of energy treatment instrument, an advantage is provided in that it is possible to uniformly raise a temperature of a treatment target tissue in biological tissue and appropriately treat the treatment target tissue.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An energy treatment instrument comprising:
a first jaw that includes a first holding surface, the first holding surface including
a first end region,
a second end region that is separated from the first end region, and
a first reference position that is located between the first end region and the second end region;
a second jaw that includes a second holding surface that holds biological tissue with the first holding surface, the second holding surface including
a third end region formed by projecting the first end region onto the second holding surface in a state in which the first holding surface and the second holding surface face each other,
a fourth end region formed by projecting the second end region onto the second holding surface in the state in which the first holding surface and the second holding surface face each other, and
a second reference position formed by projecting the first reference position onto the second holding surface in the state in which the first holding surface and the second holding surface face each other;
a first electrode that is disposed in the first end region of the first holding surface; and
a second electrode that is disposed on one of the second end region of the first holding surface and the fourth end region of the second holding surface, electrical current being applied between the first electrode and the second electrode,
wherein on the first holding surface and the second holding surface, in the state in which the first holding surface and the second holding surface face each other, a clearance between the first holding surface and the second holding surface is continuously changed toward the first reference position and the second reference position and a clearance between the first reference position and the second reference position is greatest.

2. The energy treatment instrument according to claim 1, further comprising:
a third electrode that is disposed in the other one of the second end region and the fourth end region; and
a fourth electrode that is disposed in the third end region.

3. The energy treatment instrument according to claim 2, further comprising a control device that simultaneously or time divisionally supplies high frequency electrical power between the first electrode and one of the second electrode and the third electrode and between the fourth electrode and another one of the second electrode and the third electrode.

4. The energy treatment instrument according to claim 1, wherein
the second electrode is disposed in the fourth end region,
the first holding surface includes, between the first end region and the first reference position, a first auxiliary position on the first end region side,
the second holding surface includes, between the fourth end region and the second reference position, a second auxiliary position on the fourth end region side, and
on the first holding surface and the second holding surface, in the state in which the first holding surface and the second holding surface face each other, the clearance between the first holding surface and the second holding surface at the first auxiliary position is greater than a clearance between the first end region and the third end region and the clearance between the first holding surface and the second holding surface at the second auxiliary position is greater than a clearance between the second end region and the fourth end region.

5. The energy treatment instrument according to claim 1, wherein, in the state in which the first holding surface and the second holding surface face each other, the clearance between the first reference position and the second reference position is set to be 1.5 times to 2.5 times a clearance between the first end region and the third end region and a clearance between the second end region and the fourth end region.

6. The energy treatment instrument according to claim 4, wherein, in the state in which the first holding surface and the second holding surface face each other, the clearance between the first reference position and the second reference position is set to be 1.5 times to 2.5 times a clearance between the first end region and the third end region and a clearance between the second end region and the fourth end region.

7. The energy treatment instrument according to claim 1, wherein the first reference position is located at a center between the first end region and the second end region.

8. The energy treatment instrument according to claim 1, wherein
one of the first holding surface and the second holding surface has a convex shape, and
another one of the first holding surface and the second holding surface has a concave shape.

9. The energy treatment instrument according to claim 4, wherein
one of the first holding surface and the second holding surface has a convex shape, and
another one of the first holding surface and the second holding surface has a concave shape.

10. The energy treatment instrument according to claim 1, wherein each of the first holding surface and the second holding surface has a concave shape.

11. The energy treatment instrument according to claim 2, wherein each of the first holding surface and the second holding surface has a concave shape.

12. The energy treatment instrument according to claim 4, wherein each of the first holding surface and the second holding surface has a concave shape.

13. The energy treatment instrument according to claim 4, further comprising a cooling member that is disposed at least one of electrodes between the first jaw and the second jaw, that is thermally in contact with at least one of the first electrode and the second electrode, and that cools the electrode.

14. The energy treatment instrument according to claim 13, wherein the cooling member includes a latent heat storage material.
